(19) United States
(12) Patent Application Publication
(10) Pub. No.: US 2017/0216211 A1
(43) Pub. Date: Aug. 3, 2017

(54) Aqueous Granulation Process For Amorphous Poorly Water Soluble Drugs

(71) Applicant: Sandoz AG, Basel (CH)

(72) Inventors: Franz X. Schwarz, Kundl (AT); Georg Anker, Kundl (AT); Hans Bacher, Kundl (AT); Heiko Degendorfer, Kundl (AT)

(73) Assignee: Sandoz AG, Basel (CH)

(21) Appl. No.: 15/501,312

(22) PCT Filed: Aug. 3, 2015
(86) PCT No.: PCT/EP2015/067759

§ 371 Date: Feb. 2, 2017

(30) Foreign Application Priority Data
Aug. 4, 2014 (EP) 14179750.6

(51) Int. Cl. A61K 9/16; A61K 9/20; A61K 31/501; A61K 9/28

(52) U.S. Cl. CPC A61K 9/1694; A61K 9/2893; A61K 9/2095; A61K 31/501; A61K 9/1617; A61K 9/1652

(57) ABSTRACT

The invention relates to an aqueous granulation process for preparing granules comprising an amorphous poorly water soluble active pharmaceutical ingredient, a particular polymer and a particular surfactant. The invention also refers to a process for preparing tablets comprising said granules. Furthermore, the invention refers to granules and tablets obtainable by the process of the invention.

The invention relates to an aqueous granulation process for preparing granules comprising an amorphous poorly water soluble active pharmaceutical ingredient, a particular polymer and a particular surfactant. The invention also refers to a process for preparing tablets comprising said granules. Furthermore, the invention refers to granules and tablets obtainable by the process of the invention.

### BACKGROUND PRIOR ART

Poorly water soluble pharmaceutical ingredients (in the following also “active ingredients”) are difficult to formulate because of their low solubility in water and thus slow in vivo dissolution. Providing pharmaceutical dosage forms containing such active ingredients and having a good dissolution rate is therefore challenging. Due to their better solubility, amorphous forms of said poorly water soluble active ingredients are given preference over crystalline forms. However, said amorphous forms have a tendency to crystallize during the formulation process and even during storage of the final products. Thus, said amorphous active ingredients have to be stabilized, while a good dissolution rate shall be provided at the same time.

A typical approach for formulating poorly water soluble active pharmaceutical ingredients in amorphous form is to provide a solid dispersion/solution by dissolving the active ingredient (in most cases a crystalline form) and excipients in organic solvents and preparing said solid dispersion/solution via spray drying. Solid dispersions/solutions show enhanced bioavailability due to increased solubility.

In order to prepare solid dispersions/solutions of poorly soluble active ingredients, polymers such as AQOAT of Shin-Etsu are used. For a typical method of preparation, the active ingredient and polymer (carrier) are dissolved together in a common organic solvent and the solution is spraydried or coated on some core formulations. The resulting solid is a “molecular matrix” of the polymer and the active ingredient, which demonstrates a significantly greater solubility compared to the original solubility of the drug (see http://www.elementoorganika.ru/files/aqoat.pdf).

The preparation of amorphous telaprevir (also referred to as “VX950”) as solid dispersion by applying a spray-drying process is for example described in WO 2005/123076.

Despite the fact that dosage forms of poorly water soluble active ingredients already exist, there is a need for novel dosage forms and processes for preparing same which provide benefits over or alternatives to the existing dosage forms/processes.

### SUMMARY OF THE INVENTION

It has been found that granules comprising an amorphous poorly water soluble active pharmaceutical ingredient can be prepared in the absence of organic solvents when using specific polymers as solubility enhancer and specific surfactants in a granulation process.

Avoiding the use of organic solvents allows using simple shear mixing devices, which do not have means for recovering organic solvents, and which may have no baffles. The spray dried products are fine powders with low bulk density, and often not easy to recover. For tableting, steps like dry granulation or agglomeration are required. The granules, manufactured following this invention can be used directly for tableting processes. Specifically, it is not required to use spray drying devices.

The granules prepared according to the invention are believed to provide a high dissolution rate. Unexpectedly, for example granules comprising telaprevir, prepared according to the present invention, provide a good dissolution profile that is comparable to a prior art solid dispersion of telaprevir obtained in a spray drying process by using organic solvents. For example, a complete release of the active ingredient may be achieved within 30 or 15 minutes. One advantage of the present invention is that no organic solvent(s) or solvent mixture which is able to dissolve both the active ingredient and the polymer is needed. Consequently, no process for removing such organic solvents from the product is required and no residual solvents, which are generally present if organic solvents are used, are detectable in the final product.

In addition to providing a high dissolution rate, the combination of specific polymer(s) and surfactant(s) are believed to stabilize the amorphous form of the active ingredient, for example it may prevent reagglomeration and crystallization of the amorphous active ingredient (the formation/prevention of formation of crystals can be monitored via X-ray diffraction analysis). Preventing crystallization is desirable, since a crystalline form of an active ingredient is generally less soluble than an amorphous form. Without wishing to be bound to any theory, it is believed that the intimate mixing process during the granulation of the active ingredient, polymer and surfactant provides an interactive physical mixture of active ingredient and excipients, which provides both a quick dissolution and stabilization of the active ingredient so that a crystallization of the active ingredient as well as a deterioration of solubility is prevented.

Thus, the present invention is suitable for preparing immediate release tablets/granules. In contrast, the use of surfactant alone is believed to be not sufficient for avoiding crystal growth of said active ingredient.

### DESCRIPTION OF THE FIGURES

FIG. 1

| | 0.5 | 2 | 4 |
|---|---|---|---|
| Telaprevir crystalline (Telaprevir cryst) | 0.003 | 0.003 | 0.002 |
| Telaprevir amorphous (Telaprevir amorph) | 0.040 | 0.008 | 0.005 |
| Telaprevir crystalline + SDS | 0.157 | 0.275 | 0.344 |
| Telaprevir amorphous + HPMCAS + SDS | 3.126 | 14.844 | 8.584 |
| Telaprevir amorphous + Soluplus + SDS | 2.918 | 3.418 | 3.533 |

FIG. 2

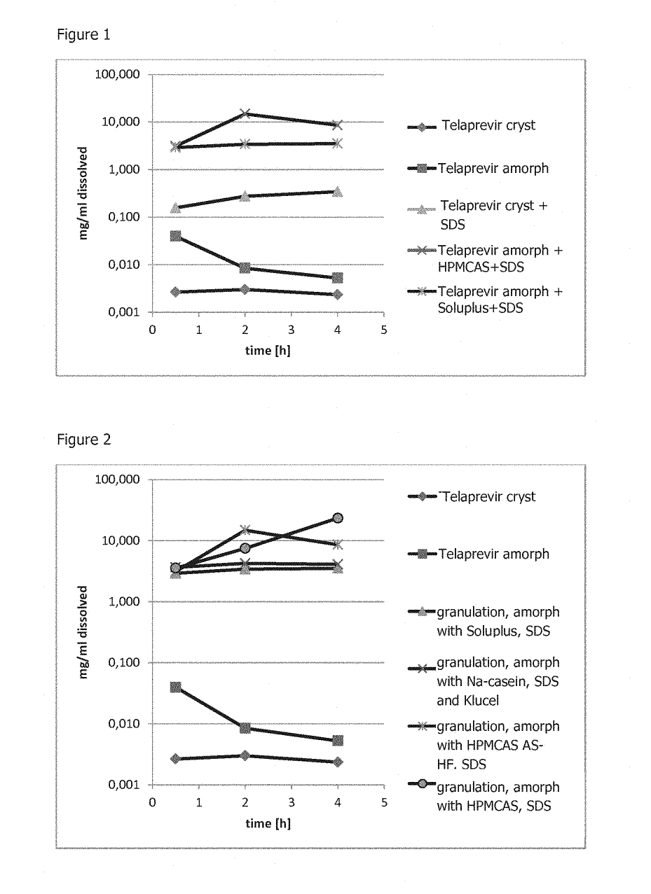

| | 0.5 h | 2 h | 4 h |
|---|---|---|---|
| Telaprevir crystalline | 0.003 | 0.003 | 0.002 |
| Telaprevir amorphous | 0.040 | 0.008 | 0.005 |
| granulation, amorphous with Soluplus, SDS | 2.9 | 3.4 | 3.5 |
| granulation, amorphous with Na-casein, SDS | 3.7 | 4.2 | 4.1 |
| and Klucel | | | |
| granulation, amorphous with HPMCAS | 3.1 | 14.8 | 8.6 |
| AS-HF, SDS | | | |
| granulation, amorphous with HPMCAS, SDS | 3.6 | 7.5 | 23.4 |

### DETAILED DESCRIPTION

The invention relates to a process for preparing granules comprising an amorphous poorly water soluble active pharmaceutical ingredient, wherein the process comprises the steps of:

(i) providing a (solid) mixture consisting of:

(a) one or more, such as for example one, two or three, preferably one or two, active pharmaceutical ingredients, wherein at least one active pharmaceutical ingredient is an amorphous particulate poorly water soluble active pharmaceutical ingredient having a solubility in water of less than 1 g/l, and 20° C.;

(b) a particulate polymer selected from the group consisting of hypromellose acetate succinates (HPMCAS, e.g. AQOAT AS-HF of Shin Etsu), polyvinyl caprolactam-polyvinyl acetate-polyethylene glycol graft copolymers (e.g. Soluplus® of BASF), polyvinylpyrrolidone, sodium-casein, hypromellose, and mixtures thereof; and

(c) optional further solid excipients;

(ii) granulating the solid mixture of step (i) by adding an aqueous solution comprising a surfactant to/onto the mixture of step (i) without thereby completely dissolving the active pharmaceutical ingredient; and

(iii) drying the granules obtained in step (ii); and

(iv) obtaining the granules comprising said amorphous poorly water soluble active pharmaceutical ingredient.

In step (i) of the process of the invention a mixture consisting of an amorphous particulate poorly water soluble active pharmaceutical ingredient, a particular polymer, a particular surfactant and optional further solid excipients is provided.

The active ingredient has a solubility in water of less than 1 g/l, preferably of less than 0.1 or 0.01 g/l, and 20° C. Such active ingredients generally have a bad wettability and pronounced tendency to crystallize.

Preferred poorly water soluble active ingredients are selected from the group consisting of telaprevir, enzalutamide, odanacatib, suvorexant, simeprevir, ritronavir, lapinavir, posaconazole, itraconazole, aprepitant, saquinavir, felodipine, ARN-509, ivacaftor, olaparib, regorafenib, vemurafeniband and nimodipine.

The polymers and surfactants that can be used in combination with the aforementioned active ingredients are described herein. It is understood that these active ingredients are all poorly water soluble, but may differ to some extent in some properties such as lipophilicity. Thus, a combination of one particular polymer and one particular surfactant that provides best dissolution results for one type of active ingredient may not necessarily be the best combination for another active ingredient. The best combination of polymer and surfactant for a particular active ingredient can be determined in routine experiments according to the examples described herein.

The active ingredient is in particulate form having a preferred particle size of 0.001-100 μm. During the process of the invention, in particular during the granulation step, no size reduction of the particulate active ingredient occurs due to the mixing. Said mixing process provides a homogenous distribution in and intensive mixing with the excipients. Furthermore, only negligible amounts of active ingredient are dissolved by the aqueous granulation liquid.

The active ingredient can be used in its “free form” as well as in the form of solvates, salts and other pharmaceutically acceptable forms.

The term “amorphous” when used for describing the active ingredient means that an X-ray powder diffraction analysis of the active ingredient or final dosage form (based on the presumption that no crystalline excipients are used), e.g. using a copper anode with Cu-Kα1,2 radiation (wavelength 0.15419 nm), only shows a broad halo.

The amount of active ingredients in the tablet can for example be 0.5-1500 mg, 100-900, or 100-600 mg, for example 375 mg.

Solubility of the active ingredient can for example be determined by measuring the amount of active ingredient in a saturated solution of active ingredient in water, e.g. by using spectroscopic measurements.

The polymers suitable for the granulation process of the invention shall enhance solubility and avoid recrystallization of the active ingredient. The polymers are selected from the group consisting of hypromellose acetate succinates (HPMCAS, e.g. AQOAT AS-HF of Shin Etsu), polyvinyl caprolactam-polyvinyl acetate-polyethylene glycol graft copolymers (e.g. Soluplus® of BASF with e.g. 13% PEG 6000/57% vinyl caprolactam/30% vinyl acetate, e.g. having a mean particle size of 340 μm), polyvinylpyrrolidone, sodium-casein, hypromellose, and mixtures thereof.

In general, all types/grades of the above polymers, e.g. all types of commercially available Aqoat (of Shin-Etsu), are suitable, wherein micronized qualities are preferred, e.g. having a mean particle size of below 1 mm or a maximum size of 2 μm. Said maximum size parameter is fulfilled, if a sieve of 2000 μm is passed by all particles. The amount of succinoyl in the different grades of commercially available Aqoat can be chosed depending on the properties of the active ingredient, i.e. depending on whether the active ingredient is more or less lipophilic.

Usually, determination of mean particle sizes is done by dynamic light scattering (analysis basis by volume), e.g. by using a Horiba LA-910 device.

Further Excipients in the Granules:

As used herein, binding agents are excipients which increase the adhesion of the active ingredients and excipients during granulation. Examples of binding agents are: sugars, such as sorbitol, glucose, fructose, disaccharides as saccharose, polysaccharides; acacia gum; cellulose derivatives, such as soluble celluloses like methylcellulose, hydroxypropylmethylcellulose and hydroxypropylcellulose; tragacanth; sodium alginate and alginate derivatives and gelatin.

As used herein, disintegrants are excipients which can take up water and swell and thus improve disintegration of a tablet or granules. Examples of disintegrants are: croscarmellose sodium; starch (paste and pre-gelatinized), such as sodium starch glycolate, sodium salt of carboxymethyl starch; methacrylic acid polymer with divinylbenzene, potassium salt, Maltodextrin; crospovidone. The amount of disintegrants, e.g. croscarmellose sodium, based on the total weight of the tablet, can for example be 5-50 wt.-%, or 5-20 wt.-%.

Preferably, no further excipients are used in addition to the polymer and surfactant.

In step (ii) of the process of the invention, the solid mixture of step (i) is granulated. In this granulation step, an aqueous solution comprising a surfactant is added, preferably sprayed, onto said solid mixture, without thereby completely dissolving the active pharmaceutical ingredient, while said mixture is mixed. Any means for wetting the solid mixture with said aqueous solution can be applied. The granulation in step (ii) is preferably performed under mixing conditions which do not substantially reduce the particle size of the amorphous particulate poorly water soluble active pharmaceutical ingredient.

According to the invention, the mass (active ingredient and polymer) is granulated by using an aqueous surfactant solution in a standard shear mixer such as DIOSNA Labormischer P1/6 of the company Diosna. Any mixer, such as a fluid-bed granulator, that for example provides similar/same mixing conditions like the device Labormischer P1/6 at a mixing speed of approximately 500 and the chopper at approximately 1000 for approximately 3 min, can be used. If, instead, a solution of the polymer was used, the amount of needed water (or organic solvent) would be much higher and a simple wet granulation would not be possible with this amount of solution. The process of the invention is not performed in a spray drying device.

The term “aqueous solution” means that the solution comprises water as the only solvent, i.e. the solution does not contain any organic or inorganic solvents.

The aqueous solution applied in step (ii) comprises an amount of surfactant of from 1 g/l to 25 g/l, such as 8 g/l, preferably consists of water and surfactant only.

The surfactant is selected from the group consisting of sodium lauryl sulfate, block copolymers of ethylene oxide and propylene oxide (poloxamer), polyoxyethylene sorbitan monooleate (e.g. Tween 20, 80), and polyethylene glycol glycerides composed of mono-, di- and triglycerides and mono- and di esters of polyethylene glycol (PEG) (e.g. Gelucire®). Sodium lauryl sulfate is preferred for use in combination with telaprevir and may also be preferred for use in combination with other active ingredients of the invention.

According to the invention, the dissolution rate of the active ingredient from the granules can be improved by choosing conditions, in particular the amount of water, to provide a partial dissolution of the polymer/active ingredient during the granulation process. The amount of water used according to the invention does neither fully dissolve the polymer nor the active ingredient.

The term “granules” as used herein refers to agglomerates of particles. Since neither the polymer nor the active ingredient is fully dissolved during the granulation process, said granules show both particles of polymer as well as particle of the active ingredient. This may be confirmed by microscope analysis, such as macroscope, microscope and or SEM (scanning microscope) analysis.

The granules obtained in step (ii) of the process can for example consist of 5 wt.-% to 90 wt.-%, preferably 25-% to 75 wt.-% of said amorphous poorly water soluble active pharmaceutical ingredient, 5 wt.-% to 90 wt.-%, preferably 25 wt.-% to 75 wt.-% of said polymer and 0.5 wt.-% to 15 wt.-%, preferably 5 wt.-% to 15 wt.-% of said surfactant. The amounts are given by weight based on the weight of the dried granules.

The granules can consist of the active ingredient, polymer and surfactant, while containing optional further excipients in an amount of 0-25 wt.-% or 0-5 wt.-%.

In step (iii), the granules are dried to reduce the residual water content, wherein the drying can be performed directly in the mixer or by using a separate fluid-bed device.

The dried granulate may, depending on the active pharmaceutical ingredient that is used, preferably not contain more than 1.9%, such as 0 to 1.9% or 0.5 to 1.9% of water. The amount of water can be determined by the method of Karl Fischer as described in K. Fischer Angew. Chemie 48, 394 (1935) or in “Die Tablette, Handbuch der Entwicklung, Herstellung and Qualitätssicherung”, Annette Bauer-Brandl, Wolfgang A. Ritschel, third edition, 2012, German language, Editio Cantor Verlag Aulendorf, Chapter 4.8.11, page 419.

The invention also refers to a process for preparing a tablet, comprising or consisting of the steps of

(a) providing granules by using a process as defined herein;

(b) mixing the granules of step (a) with further excipients;

(c) compressing the mixture of step (b) into tablets; and

(d) optionally coating the tablets obtained in step (c).

Suitable compression forces to be applied in step (IV) depend on the tablet press. The resulting resistance to crushing is e.g. 30-200 N. The outer coating as defined herein can be applied by using a coating pan, dragee vessel or a coater.

In step (d), optionally one or more coatings can be applied. For example, a gastric acid resistant coating can be performed, followed by applying an outer coating. However, it is also possible to only provide an outer coating or no coating at all.

The amount of granules in the tablet can for example be 50-95 wt.-%, preferably 85-93 wt.-%, based on the weight of the tablet. The extragranular part of the tablet can comprise excipients such as flavoring agents; lubricants in particular magnesium stearate, sweetening agents in particular aspartame, disintegrants in particular including sodium croscarmellose, fillers, and/or highly dispersed silicone dioxide.

The invention also refers to granules obtainable or obtained by the process described herein, wherein said granules contain at most small amounts of organic solvents (e.g. below 3 wt.-% or even below 1 wt.-%), and the granules contain said amorphous poorly water soluble active pharmaceutical ingredient and said polymer in the form of agglomerated particles. This means that an analysis of the granules, e.g. by SEM (scanning microscope) analysis, would show particles of polymer and particles of active ingredient. The granules are prepared without adding organic solvents in the granulation process.

The invention also relates to a tablet obtainable or obtained by the process of the present invention. The tablets thus comprise or consist of:

(i) a compressed core consisting of:

compressed granules obtainable or obtained by the process described herein, and pharmaceutically acceptable excipients in a total amount in the range of from 0 wt.-% to 50 wt.-%, such as 20 wt.-% to 50 wt.-% or 30 wt.-% to 45 wt.-%, based on the weight of the tablet,

(ii) an optional gastric acid resistant coating, in an amount in the range of from 1 wt.-% to 20 wt.-%, based on the total weight of the core and gastric acid resistant coating; and

(iii) an optional outer coating, wherein the outer coating is used in an amount in the range of from 1 wt.-% to 10 wt.-%, based on the total weight of the tablet.

The coated or uncoated tablet described herein comprises, preferably consists of granules and an extragranular phase. The amount of granules in the tablet can for example be 10-95 wt.-%, preferably 40-80 wt.-%, based on the weight of the tablet. By way of example, if telaprevir is used, the amount can be for example 70 wt.-%.

Preferably, the granules and tablets described herein provide a dissolution profile, wherein the complete amount of active ingredient is released within 30 or 15 minutes.

The granules are embedded in the extragranular phase. The extragranular phase can for example comprise disintegrants, flavoring agents, sweetening agents, fillers/diluents, glidants and/or lubricants

As used herein, disintegrants are excipients which can take up water and swell and thus improve disintegration of a tablet. Examples of disintegrants are: croscarmellose sodium; starch (paste and pre-gelatinized), such as sodium starch glycolate, sodium salt of carboxymethyl starch; methacrylic acid polymer with divinylbenzene, potassium salt, Maltodextrin; crospovidone. The extragranular phase/part of the tablet, based on the total weight of the tablet, can comprise 5-15 wt.-%, or 5-10 wt.-%, disintegrants, e.g. croscarmellose sodium, in particular 1.5 wt.-% croscarmellose sodium.

As used herein, lubricants are excipients which reduce the friction between excipients. Examples of lubricants are magnesium stearate, magnesiumfumarate, fumaric-acid, and sodiumstearylfumarate. Glidants are preferably selected from the group consisting of talc and colloidal silicone oxide, as well as mixtures thereof, preferably talc, further preferred a combination of talc and magnesium stearate is used

As used herein, fillers are excipients which increase the volume of the granules or extragranular phase. Examples of fillers are mannitol, celluloses such as microcrystalline cellulose, synthetic polymers, Ca-phosphate such as calcium hydrogen phosphate, inorganic calcium salts, maize starch, polyols and pregelatinzed starch. The amount of fillers in the granules is 0-10 wt.-%, 0-8 wt.-%, 0-6 wt.-%, 0-4 wt.-%, 0-2 wt.-% or 0-1 wt.-% by weight, based on the weight of the granules. Preferably, no fillers are present in the granules.

Other excipients are known in the art and can be chosen by a skilled person depending on their function.

Gastric acid resistant coatings and outer coatings are well known in the art. An optional outer coating can for example comprise film forming agents (such as hydroxypropylmethyl cellulose, hydroxyethyl cellulose, hydroxyethyl methyl cellulose, carboxymethylcellulose sodium, hydroxypropyl cellulose, polyethylene glycol, ethylcellulose, methacrylate (Eudragit®)), binders (such as microcrystalline cellulose), plasticizers (such as stearic acid, glycerol, propylene glycol, polyethylene glycol, phthalate esters, dibutyl sebacete, citrate esters, triacetin, castor oil, acetylated, monoglycerides, fractionated coconut oil) and optionally pigments or lakes, wherein the outer coating is typically used in an amount in the range of from 1 wt.-% to 8 wt.-%, based on the total weight of the tablet.

The tablet described herein can have a breaking notch. The breaking notch can be provided on one or more sides of the tablet. Typically the breaking notch is provided on two opposing sides of a tablet. Breaking notches are sometimes also referred to as score lines or grooves.

The dissolution rate of the tablet can be determined as follows: USP Method 2, defined in United States Pharmacopeia, current edition.

The invention also relates to a method of treating patients by administering the tablets of the invention.

The following examples describe the present invention in detail, but are not to be construed to be in any way limiting for the present invention.

### Example 1

| | |
|---|---|
| Tevir (telaprevir), amorph | 1.29 g |
| AQOAT AS-HG | 1.29 g |
| Sodium lauryl sulfate (K12PH) | 0.01 g |
| Croscarmellose Sodium Ph. Eur. | 2.41 g |
| Water for granulation | 1.49 g |

Telaprevir and AQOAT AS-HG (Shin-Etsu) were premixed in dry state in a mortar by using a pistil. Sodium lauryl sulfate was dissolved in water for granulation. The premix was wetted with this solution for 3 min. Croscarmellose Sodium Ph. Eur. was added to the wet mass, and mixed into it. The resulting mixture was granulated through a 1.5 mm sieve. The wet granulate was dried under vacuum over night at room temperature and then equalized through a 1 mm sieve.

The resulting granules shows the improved solubility of the invention (see table below)

### Example 2

| | |
|---|---|
| Telaprevir, amorph | 1.27 g |
| Sodium-casein | 1.27 g |
| Sodium lauryl sulfate (K12PH) | 0.01 g |
| Hydroxypropylcellulose Ph. Eur. | 0.07 g |
| Croscarmellose Sodium Ph. Eur. | 2.38 g |
| Water for granulation | 1.35 g |

Telaprevir and sodium-casein were premixed in dry state in a mortar by using a pistil. Sodium lauryl sulfate and hydroxypropylcellulose Ph. Eur. was dissolved in water for granulation. The premix was wetted with this solution for 3 min. Croscarmellose Sodium Ph. Eur. was added to the wet mass, and mixed into it. The resulting mixture was granulated through a 1.5 mm sieve. The wet granulate was dried under vacuum over night at room temperature and then equalized through a 1 mm sieve.

### Example 3

| | |
|---|---|
| Tevir, amorph | 1.29 g |
| Soluplus | 1.29 g |
| Sodium lauryl sulfate (K12PH) | 0.01 g |
| Croscarmellose Sodium Ph. Eur. | 2.41 g |
| Water for granulation | 1.35 g |

Telaprevir and Soluplus (BASF) were premixed in dry state in a mortar by using a pistil. Sodium lauryl sulfate was dissolved in water for granulation. The premix was wetted with this solution for 3 min. Croscarmellose Sodium Ph. Eur. was added to the wet mass, and mixed into it. The resulting mixture was granulated through a 1.5 mm sieve. The wet granulate was dried under vacuum over night at room temperature and then equalized through a 1 mm sieve.

The resulting granules shows the improved solubility of the invention (see table below)

### Example 4

| | |
|---|---|
| Tevir, amorph | 1.29 g |
| AQOAT AS-HF | 1.29 g |
| Sodium lauryl sulfate (K12PH) | 0.01 g |
| Croscarmellose Sodium Ph. Eur. | 2.41 g |
| Water for granulation | 1.35 g |

Telaprevir and AQOAT AS-HF (Shin-Etsu) were premixed in dry state in a mortar by using a pistil. Sodium lauryl sulfate was dissolved in water for granulation. The premix was wetted with this solution for 3 min. Croscarmellose Sodium Ph. Eur. was added to the wet mass, and mixed into it. The resulting mixture was granulated through a 1.5 mm sieve. The wet granulate was dried under vacuum over night at room temperature and then equalized through a 1 mm sieve.

The resulting granules shows the improved solubility of the invention (see table below).

Solubility Results:

Solubility was tested as follows:

200 mg sample was added to 10 ml test solution in a standard GC head space vial, and put on a shaker at 25° C. At the mentioned time points, a sample of 0.5 ml test solution was taken, filtered with Minisart 0.45 μm and diluted 1:1 with acetonitrile/water 60:40. This solution was used for analyses of the content by HPLC.

| mg/ml dissolved | 0.5 h | 2 h | 4 h |
|---|---|---|---|
| Telaprevir crystalline | 0.003 | 0.003 | 0.002 |
| Telaprevir amorphous | 0.040 | 0.008 | 0.005 |
| Example 3 (Soluplus) | 2.9 | 3.4 | 3.5 |
| Example 2 (Sodium-casein) | 3.7 | 4.2 | 4.1 |
| Example 4 (HPMCAS AS-HF) | 3.1 | 14.8 | 8.6 |
| Example 1 (HPMCAS AS-HG) | 3.6 | 7.5 | 23.4 |

After addition of the following ingredients (listed below) and mixing, a tablet could be formed:

| | | |
|---|---|---|
| Telaprevir granules from Examples 1 to 4 | 1.4540 | 36.35 g |
| calcium hydrogen phosphate (anhydrous) | 0.1000 | 2.50 g |
| microcrystalline cellulose (PH113) | 0.0625 | 1.56 g |
| highly dispersed silicone dioxide NF, PH. EUR. | 0.0050 | 0.13 g |
| sodiumstearylfumarate | 0.0300 | 0.75 g |

Solubility Results:

When using telaprevir, an increased solubility was found for the use of different polymers as follows:

HPMCAS AS-HG (Shin Etsu)+

HPMCAS AS-HF (Shin Etsu)+

Sodium casein (please specify)++

Soluplus (of BASF)+++

### CITED LITERATURE

WO 2005/123076.
K. Fischer Angew. Chemie 48, 394 (1935).
“Die Tablette: Handbuch der Entwicklung, Herstellung and Qualitatssicherung”, 3. edition, 2012, Editio Cantor Verlag, Aulendorf, page 419.
Brochure of Shin-Etsu “Aqoat” (2005.10), page 17, http://www.elementoorganika.ru/files/aqoat.pdf.

1. Process for preparing granules comprising an amorphous poorly water soluble active pharmaceutical ingredient, wherein the process comprises the steps of:
(i) providing a mixture consisting of:
(a) one or more active pharmaceutical ingredients, wherein at least one active pharmaceutical ingredient is an amorphous particulate poorly water soluble active pharmaceutical ingredient having a solubility in water of less than 1 g/1, and 20° C.;
(b) a particulate polymer selected from the group consisting of hypromellose acetate succinates, polyvinyl caprolactam-polyvinyl acetate-polyethylene glycol graft copolymers, polyvinylpyrrolidone, sodium-casein, hypromellose, and mixtures thereof; and
(c) optional further solid excipients;
(ii) granulating the solid mixture of step (i) by adding an aqueous solution comprising a surfactant to/onto the mixture of step (i) without thereby completely dissolving the active pharmaceutical ingredient; and
(iii) drying the granules obtained in step (ii); and
(iv) obtaining the granules comprising said amorphous poorly water soluble active pharmaceutical ingredient.

2. The process of claim 1, wherein the surfactant is selected from the group consisting of sodium lauryl sulfate, block copolymers of ethylene oxide and propylene oxide, (poloxamer), polyoxyethylene sorbitan monooleate, and polyethylene glycol glycerides composed of mono-, di- and triglycerides and mono- and di esters of polyethylene glycol (PEG).

3. The process of claim 1, wherein in step (ii) said aqueous solution comprising a surfactant is sprayed onto the solid mixture of step (i).

4. The process of claim 1, wherein the granules comprise 5 wt.-% to 90 wt.-% of said amorphous poorly water soluble active pharmaceutical ingredient, 5 wt.-% to 90 wt.-% of said polymer and 0.5 wt.-% to 15 wt.-% of said surfactant, wherein the amounts are given by weight based on the weight of the dried granules.

5. The process of claim 1, wherein the granulation in step (ii) is performed under mixing conditions which do not reduce the particle size of the amorphous particulate poorly water soluble active pharmaceutical ingredient.

6. The process of claim 1, wherein the aqueous solution applied in step (ii) comprises an amount of surfactant of from 1 g/l to 100 g/l.

7. The process of claim 1, wherein the poorly water soluble active ingredient is selected from the group consisting of telaprevir, enzalutamide, odanacatib, suvorexant, simeprevir, ritronavir, lapinavir, posaconazole, itraconazole, aprepitant, saquinavir, felodipine, and nimodipine.

8. Process for preparing a tablet, comprising the steps of
(a) providing granules by using a process as defined in claim 1;
(b) mixing the granules of step (a) with further excipients;
(c) compressing the mixture of step (b) into tablets; and
(d) optionally coating the tablets obtained in step (c).

9. The process according to claim 8, wherein the amount of granules in the tablet is 50-95 wt.-%, based on the weight of the tablet.

10. Granules obtainable by the process of claim 1, wherein the granules contain said amorphous poorly water soluble active pharmaceutical ingredient and said polymer in the form of agglomerated particles.

11. Tablet comprising or consisting of:
(i) a compressed core consisting of:
compressed granules obtainable or obtained by the process of claim 1, and
pharmaceutically acceptable excipients in a total amount in the range of from 0 wt.-% to 20 wt.-%, based on the weight of the core,
(ii) an optional gastric acid resistant coating, in an amount in the range of from 1 wt.-% to 20 wt.-%, based on the total weight of the core and gastric acid resistant coating; and
(iii) an optional outer coating, wherein the outer coating is used in an amount in the range of from 1 wt.-% to 10 wt.-%, based on the total weight of the tablet.

12. The tablet of claim 11, wherein the surfactant is selected from the group consisting of sodium lauryl sulfate, block copolymers of ethylene oxide and propylene oxide, (poloxamer), polyoxyethylene sorbitan monooleate, and polyethylene glycol glycerides composed of mono-, di- and triglycerides and mono- and di esters of polyethylene glycol (PEG).

13. The tablet of claim 11, wherein in step (ii) said aqueous solution comprising a surfactant is sprayed onto the solid mixture of step (i).

14. The tablet of claim 11, wherein the granules comprise 5 wt.-% to 90 wt.-% of said amorphous poorly water soluble active pharmaceutical ingredient, 5 wt.-% to 90 wt.-% of said polymer and 0.5 wt.-% to 15 wt.-% of said surfactant, wherein the amounts are given by weight based on the weight of the dried granules.

15. The tablet of claim 11, wherein the granulation in step (ii) is performed under mixing conditions which do not reduce the particle size of the amorphous particulate poorly water soluble active pharmaceutical ingredient.

16. The tablet of claim 11, wherein the aqueous solution applied in step (ii) comprises an amount of surfactant of from 1 g/l to 100 g/l.

17. The tablet of claim 16, wherein the aqueous solution applied in step (ii) consists of water and surfactant.

18. The tablet of claim 11, wherein the poorly water soluble active ingredient is selected from the group consisting of telaprevir, enzalutamide, odanacatib, suvorexant, simeprevir, ritronavir, lapinavir, posaconazole, itraconazole, aprepitant, saquinavir, felodipine, and nimodipine.

19. The process of claim 1, wherein the granules consist of 5 wt.-% to 90 wt.-% of said amorphous poorly water soluble active pharmaceutical ingredient, 5 wt.-% to 90 wt.-% of said polymer and 0.5 wt.-% to 15 wt.-% of said surfactant, wherein the amounts are given by weight based on the weight of the dried granules.
